(19) United States
(12) Patent Application Publication
(10) Pub. No.: US 2010/0210942 A1
(43) Pub. Date: Aug. 19, 2010

(54) SYSTEM AND METHOD FOR ULTRASOUND ANALYSIS OF BIOLOGICAL STRUCTURES

(75) Inventors: Timothy M. Chang, Montville, NJ (US); Kavil Patel, Mumbai (IN)

Correspondence Address: GIBSON & DERNIER LLP, 900 ROUTE 9 NORTH, SUITE 504, WOODBRIDGE, NJ, 07095, US

(73) Assignee: NEW JERSEY INSTITUTE OF TECHNOLOGY, Newark, NJ (US)

(21) Appl. No.: 12/625,078

(22) Filed: Nov. 24, 2009

Related U.S. Application Data
(60) Provisional application No. 61/118,261, filed on Nov. 26, 2008.
(60) Provisional application No. 61/121,322, filed on Dec. 10, 2008.

(51) Int. Cl. A61B 8/00

(52) U.S. Cl. 600/437

(57) ABSTRACT

Ultrasound systems and methods for detecting features of biological tissue, including fracture detection in bone tumor detection are described. Systems and methods may employ low transient pulse technology. Methods employ detection and analysis of behavioral patterns of different signal parameters such as flight time, maximum amplitude, phase angle, and correlation as indicators of the depth of a fracture and may be used in quantitative ultrasound bone fracture detection. Flight time and frequency components may be employed in tumor detection methods.

### CROSS REFERENCE TO RELATED APPLICATIONS

This application claims the benefit of U.S. Provisional Patent Application Ser. Nos. 61/118,261, filed Nov. 26, 2008 and 61/121,322, filed Dec. 10, 2008, the entireties of which are incorporated by reference herein in their entireties.

### FIELD OF THE INVENTION

This invention relates to ultrasound apparatus, systems and methods for detection and quantitative analysis of biological structures, particularly to analysis of skeletal and soft tissue structures using low transient pulse technology.

### BACKGROUND OF THE INVENTION

Fatigue fractures, also known as stress fractures, are one type of incomplete fracture in bone. Fatigue fractures are often seen as fine disruptions of normal bone architecture. These micro fractures are caused due to an activity which exerts repetitive sub-threshold loading which over time exceeds bone's intrinsic ability to heal itself, for example running or jumping. Morris, “Fatigue fractures”, Calif. Med. 108(4): 268-74 (1968). Fatigue fractures are commonly associated with athletes who run and jump on hard surfaces, such as distance runners, basketball players, and ballet dancers. Fatigue fractures are also known to occur in a deconditioned person who just started a new exercise program, such as military recruits. Monteleone, “Stress fracture in the athlete” Ortho. Clin. North Am., 26: 423-32 (1995); Maitra et al., “Stress Fractures, Clinical history and physical examination” Clin. Sports Med., 16: 259-74 (1997); Beck, “Tibial stress injuries: An aetiological review for the purposes of guiding management” Sports Med., 26(4): 265-79 (1998). Fatigue fractures commonly occur in lower limbs as a result of the ground reaction forces that must be dissipated during running, walking, marching or jumping. Stress related fractures in tibia account for approximately one-half of all stress fractures in children and adults, and fatigue fractures in metatarsals represent approximately 25% of stress fractures. Bennell, et al., “Epidemiology and site specificity of stress fractures.” Clin. Sports Med., 16(2): 179-96 (1997); Brukner, “Exercise-related lower leg pain: bone.” Med. Sci. Sports Exerc., 32(3 Suppl): S15-26 (2000). In adults approximately 10% and in children about 20% of fatigue fractures occur in the distal end of fibula. Coady et al., “Stress fracture in the pediatric athlete” Clin. Sports Med. 16: 225-38 (1997).

Currently the most common methods of detecting fractures in bone are x-ray, MRI and CT. However, current technology used in detecting fatigue fractures is often unreliable, difficult and costly. Swischuk et al., “Frequently missed fractures in children (value of comparative views)” Emerg. Radiol., 11(1): 22-8 (2004). Misdiagnosis in fracture detection partly arises from fractures that are simply difficult to see with x-rays. Many types of fractures are easily missed in children. The presence of a fatigue fracture may not show up on plain radiograph film for up to 2 to 10 weeks after symptom onset, which increases the likelihood of a nonunion. Maitra et al., “Stress fractures, clinical history and physical examination” Clin. Sports Med., 16: 259-74 (1997). A fracture that may be unseen on a single x-ray may be revealed when several x-rays from multiple vantage points and different angles are taken. However, using multiple x-rays at different vantage points to detect fatigue fractures requires the patient to to absorb increased amounts of harmful radiation. The inability and uncertainty in detecting fatigue fractures using plain radiograph films leave physicians to resort to costlier methods, such as bone scan, MRI or CT. Using bone scan, MRI or CT to carry out the diagnosis increases the cost of the treatment.

Current bone fracture detection methods such as x-rays, CTs and MRIs are all qualitative as they require interpretation by highly trained and experienced physicians. Although these tests can be performed repeatedly throughout the fracture life span, the cost of the treatment and the time spent by physicians and technicians interpreting the data makes such an approach prohibitive.

Along with the cost and time required for qualitative methods there exists the possibility of errors in the readings. These errors can be induced due to physicians' technique, perception, knowledge, judgment or communication. Fitzgerald, “Error in radiology” Clin. Radiol., 56(12): 938-46 (2001). Along with the errors, there are also differences in diagnoses among physicians. The qualitative nature of the tests is responsible for all the induced errors. These errors can be decreased by providing physicians with quantitative results.

The use of Quantitative Ultrasound System (QUS) has become more widespread in the field of orthopedics due to its potential advantages over CT, MRI, and X-ray in terms of cost, size, safety, and detection resolution. Frost et al. “A comparison of fracture discrimination using calcaneal quantitative ultrasound and dual X-ray absorptiometry in women with a history of fracture at sites other than the spine and hip” Calcif. Tissue Int., 71(3): 207-11 (2002). A number of studies have been conducted to investigate the use and reliability of QUS in detecting osteoporosis in patients. An underlying principle behind these ultrasound devices is to monitor the velocity of sound in bone, which is governed by the thickness of the cortical bone. In these devices only one parameter of acoustic signal is investigated, thus the signal cannot be quantified properly to yield a proper diagnosis. Sarvazyan et al., “Application of the dual-frequency ultrasonometer for osteoporosis detection” Ultrasonics (2008).

Similarly, soft tissue analysis, detection and screening methods, such as those used for tumor detection, including x-ray (mammography), ultrasound and CT are expensive, inconvenient to use, and relatively unsafe due to their ionizing energy. Moreover, at present, the usage of ultrasound is primarily for guiding surgical procedures when tumor features have already been identified. All of the prior art methods are based on visual examination and therefore require extensive time/effort and are subject to interpretation errors.

Another major consideration in ultrasound imaging in bone and soft tissue is resolution quality. Traditionally, in order to achieve high enough resolution, the operating frequency must be increased, into the tens of megahertz range, thereby increasing the cost of instrumentation while reducing the depth of sound penetration and the amount of tissue information. Furthermore, successive signal packets tend to interfere with each other due to transducer transients which further limit the detection resolution. Pulse aliasing is a major problem: different echoes combine to form a single echo signal, thereby obscuring true signal characteristics (flight time, amplitude, etc.).

Thus there is a need for diagnostic devices and methods which can be used at low cost, provide quantitative information for analyzing, diagnosing and/or monitoring bone fractures and soft tissue with a decreased dependency on experts to interpret the results and which would reduce variance and increase accuracy. Moreover, there is a need for such devices and methods which reduce the exposure of patients to harmful radiation.

### SUMMARY OF THE INVENTION

It is an object of the present invention to provide an improved system for analyzing biological tissue using ultrasound.

In accordance with one embodiment a system is provided for detecting and monitoring fatigue fractures and other structural damage and/or defects in bone.

In accordance with another embodiment a system is provided for analyzing bone includes a signal generator, a transmitter, one or more receivers and a signal analyzer. The signal generator is preferably one which can generate a Low Transient Pulse (LTP) signal.

In an exemplary embodiment, the system may include a Quantitative Ultrasound System (QUS) detection system employing a LTP signal generator.

In accordance with a further embodiment a method of analyzing bone is provided comprising essentially generating a signal to a transmitter, applying the transmitter and one or more receivers to a body structure containing the bone to be analyzed, transmitting the signal to the bone to be analyzed, and transmitting a received signal to a signal analyzer. In a preferred embodiment, the method employs generating a LTP signal.

In a further embodiment, the detection method employs both time and frequency domain interpretation. The method may employ a LTP signal where within each period of pulse excitation, the entire pulse shape is modulated by a single random number. Over multiple periods, the pulse amplitudes are randomized so that there is enough passband energy to cover resonances and the changes induced by the fracture.

The method may use axial QUS techniques, using unilateral positioning of transducers at available sites of long bones covered by a thin layer of soft tissues. In a preferred embodiment the method includes applying a transmitter and receivers in positions to allow the capture of ultrasound surface waves propagating along the bone axis, permitting the analysis of the compact bone of the cortex.

The method may include evaluation of signal parameters selected from amplitude, RMS energy, flight time and correlation analysis of the received acoustic signal. A fracture typically induces attenuation of amplitude and energy while flight time increases. These effects are most notable when the fracture depth is large compare to the wavelength of the ultrasound signal. For fracture depth less than a wavelength, change of resonant frequency may be employed.

In a preferred embodiment a bone fracture detection method is provided which employs a low transient pulse drive signal and analysis of flight time, maximum amplitude, phase change, and correlation analysis of the received acoustic signal.

In another embodiment a bone fracture detection method is provided in which the placement of transducers provides a reference reading which is unaffected by the fracture and a reading which is affected by the fracture. The reference reading makes the injured bone act as its own calibration standard. Using the behavioral patterns of the signal parameters flight time, maximum amplitude, phase change, and correlation analysis of the received acoustic signal, an algorithm is generated which determines the fracture condition by comparing signal parameters from R1 to signal parameters of R2.

It is a further object of the present invention to provide a system for detecting tumors.

In accordance with one embodiment the inventive system solves the problems encountered in current devices with respect to tumors in general and breast cancer tumors specifically, and includes a signal generator, a transmitter, one or more receivers and a signal analyzer. The signal generator is preferably one which can generate a Low Transient Pulse (LTP) signal. A transmitter and at least one receiver are used for detection of ultrasonic signals, wherein the receivers are operated in the same way as a stethoscope. The reduction in transducer count enables compact, low cost electronics to be used. Use of a second receiver ratiometrically removes uncertainties due to patient variability and as such contact pressure and surface condition. The use of optional low transient pulse reduces pulse coupling. See, Chang et al., “Motion Control Firmware for High Speed Robotic Systems,” IEEE Transactions on Industrial Electronics, vol. 25, pp. 1713-1722, 2006; B. Cheng and T. N. Chang, “Enhancing Ultrasonic Imaging with Low Transient Pulse Shaping,” IEEE Trans. on Ultrasonics, Ferroelectrics, and Frequency Control, vol. 54, pp. 627-635, March 2007, incorporated herein by reference in their entireties.

It is another object of the present invention to provide a method of quantitative ultrasound tumor detection employing suitably synthesized ultrasound signals which safely penetrates and analyzes different tissue layers.

In accordance with one embodiment a method of quantitative ultrasound tumor detection includes interpretation of echo signals to infer tissue characteristics directly, without subjective interpretation. The newly developed quantitative ultrasonography differs from conventional ultrasound systems at least in that the sonic signals are numerically analyzed to identify abnormal features. Due to differences in density and elastic modulus, signals transmitted through or reflected from a tumor exhibit changes in spectral characteristics (such as frequency dependent magnitude and phase) and scattering. Together with feature size, left-right differentiation, and growth characteristics, a rapid and effective first line diagnosis can be made so that those at risk of developing cancer such as breast cancer can be referred to second line screening proactively via one embodiment of the present invention. For example, data obtained using the 150 KHz laboratory quantitative ultrasound system indicates that features size of 5-10 mm can be readily resolved. In order to minimize the aliasing of sonic signals due to multi-path, multi-target reflections, the low transient pulse method may be applied to generate very short duration sonic signals to achieve a better spatial resolution and signal to noise ratio.

### BRIEF DESCRIPTION OF THE DRAWINGS

So that those having ordinary skill in the art will have a better understanding of how to make and use the disclosed systems and methods, reference is made to the accompanying figures wherein:

FIG. 1 depicts a block diagram of a system in accordance with at least one embodiment of the present invention;

FIG. 2 illustrates a Low Transient Pulse design scheme in accordance with at least one embodiment of the present invention;

FIG. 3 graphically depicts Type-0 (left) and Type-1 (right) LTP drive signals in accordance with at least one embodiment of the present invention;

FIG. 4 is a schematic representation of a first transducer placement setup configuration in accordance with at least one embodiment of the present invention;

FIG. 5 is a schematic representation of a second transducer placement setup configuration in accordance with at least one embodiment of the present invention;

FIGS. 6A and 6B are diagrammatic representations of an isometric (6A) and a cross section (6B) view of sheep tibia in accordance with at least one embodiment of the present invention;

FIG. 7A is a graphical depiction of a receiver signal using regular rectangular pulse as the drive signal in accordance with at least one embodiment of the present invention;

FIG. 7B is a graphical depiction of a receiver signal using Type-0 LTP (one pulse method) as the drive signal in accordance with at least one embodiment of the present invention;

FIG. 7C is a graphical depiction of a receiver signal using Type-1 LTP (two pulse method) as the drive signal in accordance with at least one embodiment of the present invention;

FIG. 8A is a graphical depiction of coefficient of variation of LTP and RRP in the amplitude at R1 in accordance with at least one embodiment of the present invention;

FIG. 8B is a graphical depiction of coefficient of variation of LTP and RRP in the amplitude at R2 in accordance with at least one embodiment of the present invention;

FIG. 8C is a graphical depiction of coefficient of variation of LTP and RRP in the flight time at R1 in accordance with at least one embodiment of the present invention;

FIG. 8D is a graphical depiction of coefficient of variation of LTP and RRP in the amplitude at R2 in accordance with at least one embodiment of the present invention;

FIG. 9A is a schematic representation of a transducer placement setup configuration in accordance with at least one embodiment of the present invention;

FIG. 9B is a schematic representation of a transducer placement setup configuration in accordance with at least one embodiment of the present invention;

FIG. 10A is a graphical depiction of flight time of the received acoustic signal at R2 in Setup 1 with respect to fracture depth in accordance with at least one embodiment of the present invention;

FIG. 10B is a graphical depiction of flight time of the received acoustic signal at R2 in Setup 2 with respect to fracture depth in accordance with at least one embodiment of the present invention;

FIG. 11A is a graphical depiction of maximum amplitude of acoustic signal captured by R2 in Setup 1 vs. fracture depth in accordance with at least one embodiment of the present invention;

FIG. 11B is a graphical depiction of maximum amplitude of acoustic signal captured by R2 in Setup 2 vs. fracture depth in accordance with at least one embodiment of the present invention;

FIG. 11C is a graphical ratiometric (R2/R1) depiction of maximum amplitude of Setup 1 vs. fracture depth in accordance with at least one embodiment of the present invention;

FIG. 11D is a graphical ratiometric (R2/R1) depiction of maximum amplitude of Setup 2 vs. fracture depth in accordance with at least one embodiment of the present invention;

FIG. 12A is a graphical depiction of correlation coefficients of received signals at various fracture depths in Setup 1 in accordance with at least one embodiment of the present invention;

FIG. 12B is a graphical depiction of correlation coefficients of received signals at various fracture depths in Setup 2 in accordance with at least one embodiment of the present invention;

FIGS. 13A and 13B depict a top (13A) and side (13B) view schematic representation of a transducer placement setup configuration in accordance with at least one embodiment of the present invention;

FIG. 14 depicts a block diagram of a system in accordance with at least one embodiment of the present invention;

FIGS. 15A and 15B contain graphical depictions of data from breast phantom: FIG. 15A, control, no tumor; FIG. 15B, presence of 1 cm tumor, in accordance with at least one embodiment of the present invention;

FIGS. 16A and 16B contain graphical depictions of spectral characteristics for tumor size differentiation: FIG. 16A, 1 cm tumor; FIG. 16B, 2 cm tumor, in accordance with at least one embodiment of the present invention;

FIG. 17A is a graphical representation of an axial ultrasound signature without tumor in accordance with at least one embodiment of the present invention;

FIG. 17B is a schematic of transducer placement resulting in the ultrasound signature in FIG. 17A in accordance with at least one embodiment of the present invention;

FIG. 17C is a graphical representation of an axial ultrasound signature with tumor in accordance with at least one embodiment of the present invention;

FIG. 17D is a schematic of transducer placement resulting in the ultrasound signature in FIG. 17C in accordance with at least one embodiment of the present invention;

FIGS. 18A and 18B are graphical representations of echo (FIG. 18A) and spectral characteristics (FIG. 18B) corresponding to 1 cm feature size in accordance with at least one embodiment of the present invention; and

FIGS. 18C and 18D are graphical representations of echo (FIG. 18C) and spectral characteristics (FIG. 18D) corresponding to 2 cm feature size in accordance with at least one embodiment of the present invention.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

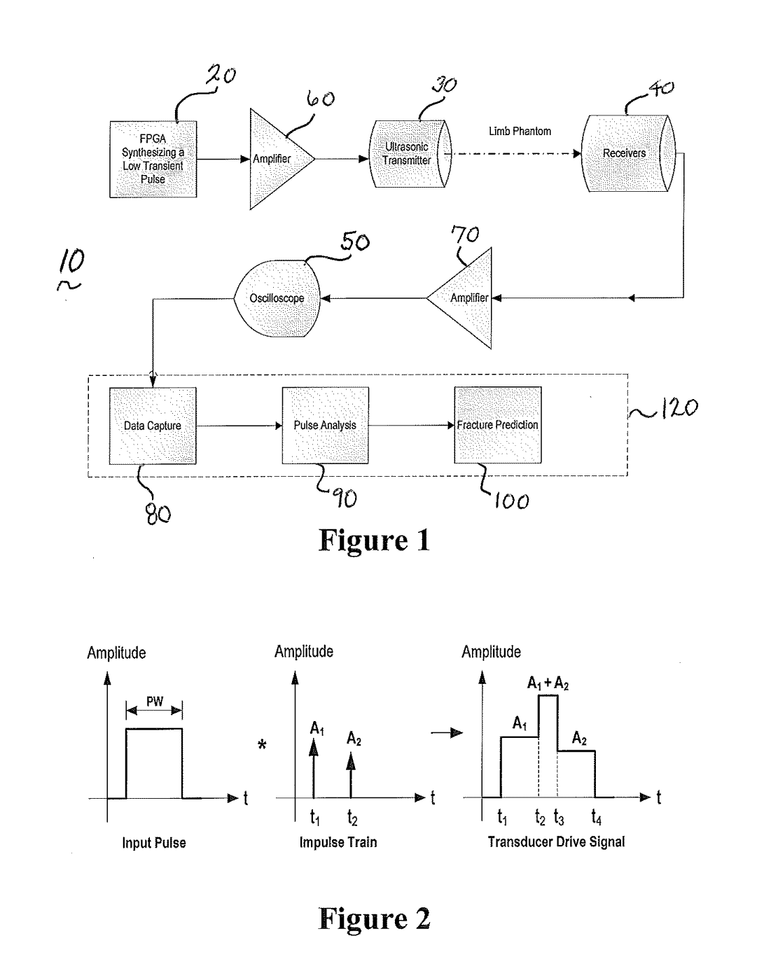

Now referring to FIG. 1 a system in accordance with at least one embodiment of the present invention is depicted. System 10 includes a signal generator 20, transmitter 30, receivers 40, and signal analyzer 50. The system may further include amplifiers 60 and 70. The transmitter 30 and receivers 40 are positioned on the biological tissue S to be analyzed.

Signal generator 10 is any signal generator which can produce a suitable signal. In accordance with one embodiment, a Field Programmable Gate Array (FPGA) may be programmed to synthesize and deliver a low transient pulse (LTP) which can be used as a drive signal for an ultrasonic transducer such as transmitter 30. An ultrasonic pulse is transmitted by transmitter 30 and received by receivers 40 placed on a specific specimen S. The signal analyzer 50 such as a high bandwidth digital oscilloscope is used to capture the high frequency ultrasound signals. As shown in blocks 80, 90 and 100, these captured signals are then analyzed and processed such as in a computer analyzer 120 to obtain essential parameters which help in fracture prediction.

By way of example, a FPGA was used in experiments described below as the signal generator 10 to produce LTP drive signals for the transducers, i.e., transmitter 30 and receivers 40. A digital signal processing and data acquisition board (Dalanco-Spry model AVR-32) was used to generate the LTP. The board was a PCI bus, PC add-in card powered by a Digital Signal Processor (DSP)(Texas Instruments TMS320C32) and a FPGA (Xilinx Spartan). The configuration of the FPGA establishes the interaction of the D/A converter, and digital I/O with the local bus. For the experiments involving this embodiment of the present invention the sampling rate of the D/A converter can be set by the timer in the DSP. The clock frequency of the FPGA was 62.5 MHz. For this study the combination of DSP and FPGA was used to synthesize a LTP drive signal. The combination of DSP and FPGA along with FPGA's high clock frequency generates a LTP drive signal with a high temporal resolution. The combination also provides flexibility in pulse design which makes configuration of LTP according to specimen characteristics an efficient process. The incrementing counter value at every clock frequency defines the pulse width of the LTP. Pulse width of the LTP drive signal is the only input required by the user. The pulse width of the drive signal was determined based on the frequency spectrum of the specimen. Running the program generates the pre-defined LTP.

LTP technology, described in detail in U.S. Pat. No. 7,492,668, the entirety of which is incorporated herein by reference, compresses acoustic pulses to pre-specified short durations. The LTP technology as used in the examples and experiments discussed herein essentially produces a short duration and low transient acoustic pulse by means of pre-shaping the excitation signal. The present inventors have verified that employing LTP technology produces a better measurement resolution and simpler hardware implementation due to less phase interference and a less complex algorithm. No modulation circuits or regenerative loops are necessary to synthesize the drive signal.

Within the quantitative ultrasonography context, employing LTP techniques improves detection resolution by minimizing aliasing of signals transmitted from soft and hard tissues. This improvement is achieved by convolving the input pulse width and impulse sequence to generate a necessary drive signal. FIG. 2 shows a pulse-impulse convolution scheme.

The transducer drive signal so produced is then stored in a computer and later fed into the ultrasound transmitter 30. These impulse parameters are obtained by considering the transducer up to the first resonance using a second order system as shown:

$$G ( s ) = \frac{α s}{{s}^{2} + 2 {ζω}_{n} s + {ω}_{n}^{2}}$$

The design parameters of a two-impulse low transient pulse shaper can be obtained as follows:

$$Δ T = \frac{n π}{{ω}_{n} \sqrt{1 - {ζ}^{2}}} = \frac{n π}{{ω}_{d}} , {M}_{p}^{n} = {( {e}^{\frac{ζπ}{\sqrt{1 - {ζ}^{2}}}} )}^{n} , n = 1 , 3 , 5$$

where π/ω_{d} is the half-ringing period of the transducer. For the two-impulse case, the desired number of oscillation cycles of the acoustic signal is determined by n. Once n is determined, ΔT and Mₚⁿ can be readily determined either from experimental measurements of the transmitter characteristics or from the damping ratio ζ and natural frequency ωₙ of the transmitter.

A Type-1 LTP drive signal is a transmitter optimized LTP drive signal which will produce a low transient pulse on the transmitter outputs. This is achieved by properly selecting the pulse width so that the transients are cancelled at the transmitter side. The design parameters are given as follows:

$$PW = Δ T , {t}_{1} = 0 , {t}_{2} = Δ T , {t}_{3} = 2 Δ T$$

$${A}_{1} = \frac{1}{1 + {M}_{p}^{n}} , {A}_{2} = \frac{{M}_{p}^{n}}{1 + {M}_{p}^{n}}$$

A Type-0 LTP drive signal is considered to be a special case of the design algorithm discussed above. The Type-0 LTP drive signal is formed by setting the parameters to the following values:

$${t}_{1} = 0 , \\ {A}_{1} = 1 , {A}_{2} = 0 , \\ PW = {t}_{2} = k \frac{2 π}{{ω}_{d}} , k = 1 , 2 , 3 , …$$

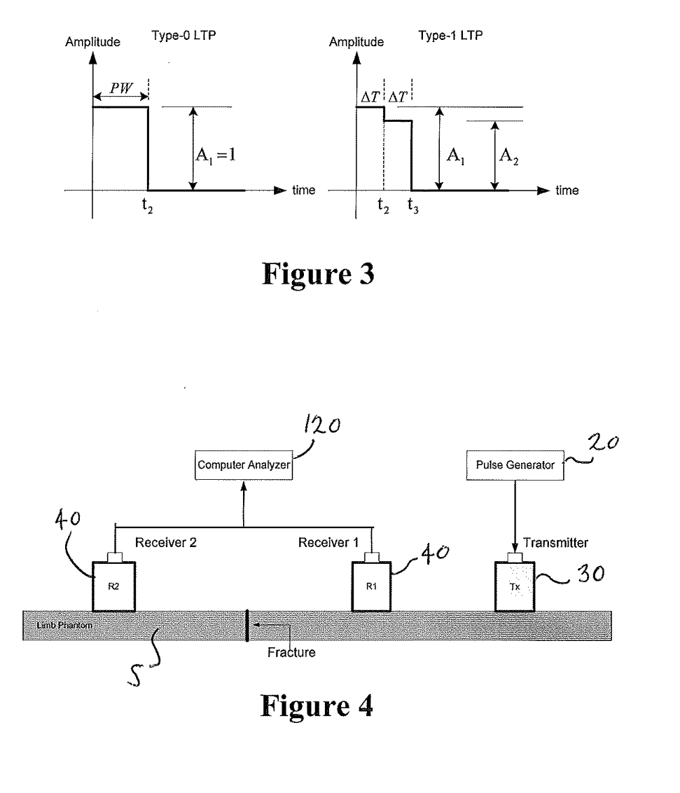

The performance of a Type-0 LTP drive signal depends on the damping of the ultrasonic transducer. Generally, a lower damping results in a higher performance. Typical Type-0 LTP and Type-1 LTP drive signals are shown in FIG. 3. In the examples and experiments below both Type-0 and Type-1 LTP drive signals were synthesized and the performance associated to each drive signal evaluated.

Transmitter 30 and receivers 40 may be any transducers suitable for use in quantitative ultrasound. For example, in the experiments below, general purpose R15a Frequency Acoustic Emission Sensors manufactured by Physical Acoustic Corporation were used. The active element of the R15a acoustic transducer is piezoelectric ceramic, may have a resonant frequency of 150 KHz and an operating frequency which ranges from 50-200 KHz. This transducer has a stainless steel outer casing and ceramic face plate which contacts the specimen S. These transducers weigh 34 grams and are 19 mm in diameter and 22.4 mm in height. In this embodiment the transducers may be used as transmitter 30 as well as receivers 40.

In one embodiment three transducers may be used—one transmitter 30 and two receivers 40. Input/output calibration can be performed to measure frequency response of the transducers. The transducers can be arranged in direct coupling and a sine wave used as a drive signal. The amplitude of the output signal at each frequency can be recorded and the results plotted to show the frequency response of each transducer. In this way the frequencies and acoustic characteristics of the transducers can be matched.

Amplifiers 60 and 70 are any suitable acoustic amplifiers, such as for example an AE2A/AE5A wide bandwidth acoustic emission amplifier.

### Experiments—Bone

Experiments were carried out on sheep tibia simulating different fracture depths to prove the ability of ultrasound to detect fractures in an actual physiological environment. Transducers were mounted on the bone surface with synthetic bee wax glue. Bee wax glue provided stability and ensured that the transducers did not move from their mounting position, providing a fixed distance between each transducer through the course of the experiments. This method also achieved a flush contact surface between the transducer faceplate and bone.

The axial quantitative ultrasound method used in the experiments, QUS, does not use ultrasonic reflection to characterize a material but passes the ultrasonic energy along a material and quantifies the effects the material has on the energy. Hubner et al., “Ultrasound in the diagnosis of fractures in children.” J. Bone Joint Surg. Br., 82(8): 1170-3 (2000); Knapp et al., “Can the WHO definition of osteoporosis be applied to multi-site axial transmission quantitative ultrasound?” Osteoporos. Int., 15(5): 367-74 (2004), both incorporated by reference herein in their entireties. QUS is described in Tatarinov et al., “Use of multiple acoustic wave modes for assessment of long bones: model study” Ultrasonics, 43(8): 672-80 (2005), incorporated herein by reference in its entirety. See also, Lowet et al., “Ultrasound velocity measurement in long bones: measurement method and simulation of ultrasound wave propagation.” J. Biomech., 29(10): 1255-62 (1996); Camus et al., “Analysis of the axial transmission technique for the assessment of skeletal status.” J. Acoust. Soc. Am., 108(6): 3058-65 (2000), both of which are incorporated by reference herein in their entireties.

Transducer placement in this technique is a significant factor. Transducer placements are the different combinations of distance between the transmitter, the receiver and their relation to the known fracture location. Now referring to FIG. 4 three transducers are shown on a limb phantom S. During the course of the experiments, two different setup configurations (Setup 1 and 2) were used. As shown in FIG. 4, in Setup 1 the first transducer is the transmitter (Tx) 30 and the received energy is recorded from two locations, receivers 40 (R1 & R2) along the test sample. The reason for this setup is to produce a reference recording and test recording. In the first recording (R1), the reference is taken across a known healthy uninjured, section of bone. The second recording (R2) is taken from across the suspected fracture location. The effect is that the reference recording is the signal affected by all individual local characteristics of the sample. The test recording taken across the fracture will be similarly affected by these same specific characteristics plus the characteristics of the fracture. Therefore, comparisons between the signals across the reference region and the injured region will only differ by the presence of the fracture and its status.

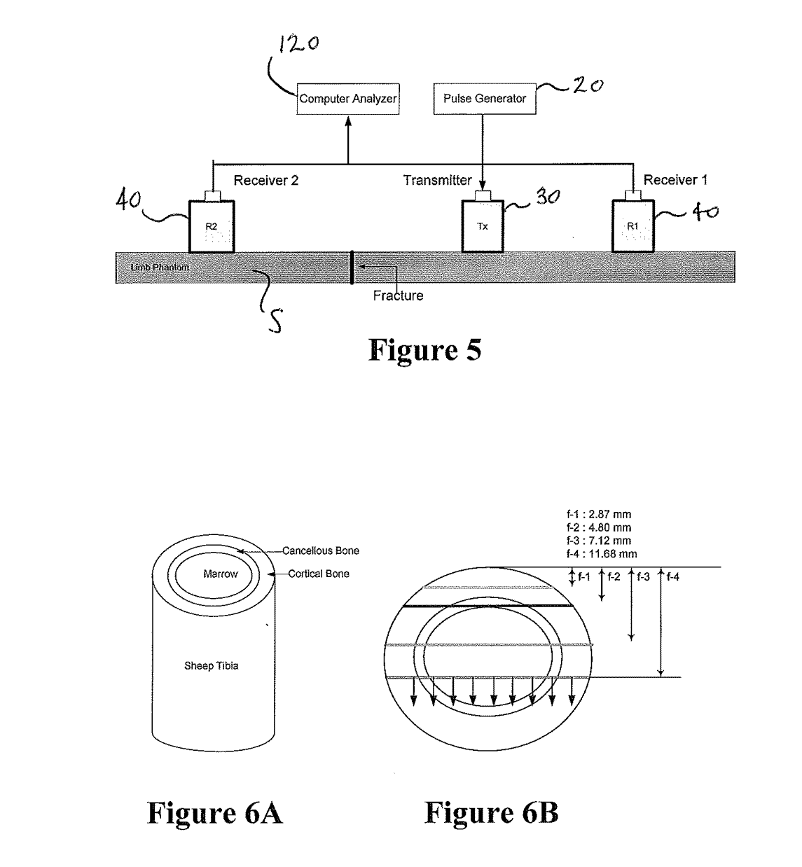

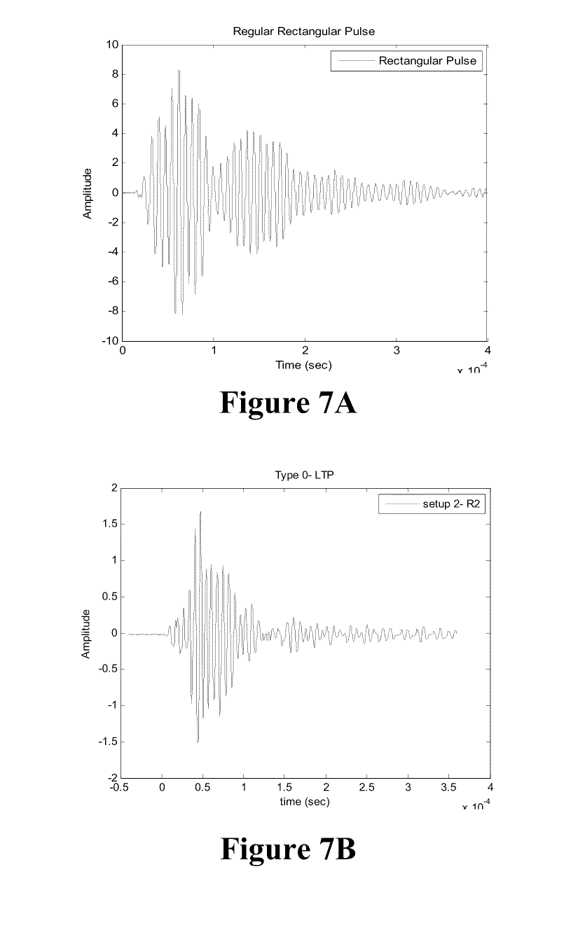

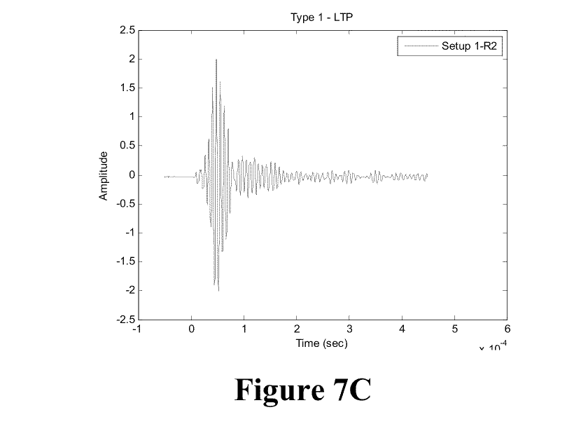

Now referring to FIG. 5, Setup 2 follows the same layout as Setup 1 except the order in which the transmitter and receivers are placed. In Setup 2 the transmitter is placed between the two receivers. One of the receivers (R1) is placed on the unaffected side of the bone and the second receiver (R2) is placed across the fracture. This configuration, in conjunction with the first setup, gives added information regarding the fracture parameters.

The distance between transducers for both setup configurations is varied based on the experiment being performed. Varying the distance between the transducers allows investigating the effects of distance on the passage of energy through the limb phantom. Acoustic energy travels at speeds dependent on different material properties. The most significant factors are the density and elastic properties of the materials. Therefore, energy packets traveling through bone and through soft tissue will be separated and arrive at the receivers at different time intervals. This separation in time intervals allows investigation of energy packets that have traveled through only soft tissue and energy packets that have only passed through bone.

Now referring to FIGS. 6A and 6B, fractures or discontinuity were simulated in sheep tibia with depth of 2.87 mm, 4.80, 7.12 mm and 11.68 mm. In FIG. 6B each line defines a fracture depth. A thin layer of glue is applied below the line to obtain the desired fracture depth. A technique was designed to simulate a hairline fracture in sheep tibia which closely represents actual fracture seen in a physiological setting. This technique involved cutting the sheep tibia in two pieces at the center point of diaphysis using a thin electric band saw, polishing the cut edges and re-attaching them with different amount of super glue each time. To study whether the acoustic properties of super glue match the acoustic properties of the bone, a cut tibia was re-attached with application of super glue throughout the cross section of cortical and cancellous bone. It was ensured that only thin uniform film of super glue was applied along the cross section. The acoustic signal captured by two receivers from an intact bone was compared to the acoustic signal captured from bone re-attached with super glue throughout its cross section. The results confirmed that material density and damping coefficient of super glue match closely to the density of the bone because no major differences were observed in signal characteristics between each case. The joined pieces of tibia were easily de-attached by applying shear force at the site of connection. Readings were then taken from the two mounted receivers in both setups after the bone was re-attached each time with different controlled levels of super glue. Cross section area where glue was applied is considered to be intact bone and area without glue in between simulates fracture. Different amount of glue yields fracture with different depth. Good care was taken during data collection to assure that the mounted transducers do not come off during the process of separating and re-attaching pieces.

Preliminary Drive Signal Comparison

Prior to the fracture measurement, the ultrasonic transducers were first tested and the corresponding drive signals configured. A comparison between low transient pulse and regular rectangular pulse was also made. Given the transducer characteristics, the following drive signals were considered:

Regular Rectangular Pulse (RRP)

Type-0 LTP

Type-1 LTP

All drive signals were synthesized by the hybrid architecture of a DSP and FPGA. The regular rectangular pulse train was designed with parameters of 2500 μs pulse period and 8.75 μs pulse width. Design parameters for the Type-0 LTP were PW=t₂=7.68 μs. Those of Type-1 LTP are synthesized with PW=ΔT=3.625 μs, A₁=0.5271 and A₂=0.4792. Now referring to 7A-7C the received signal is depicted responsive to each drive signal. It can be seen that the LTP drive signals produced a shorter transient and cleaner signal, compared to that driven by the RRP. Type-1 LTP was also successful in reducing peaks observed in the secondary reflection signals in the output.

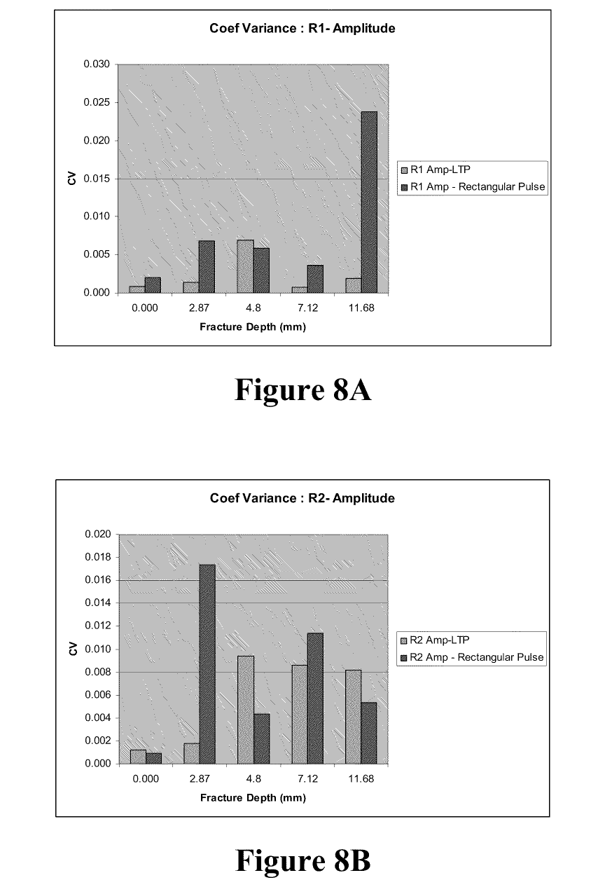

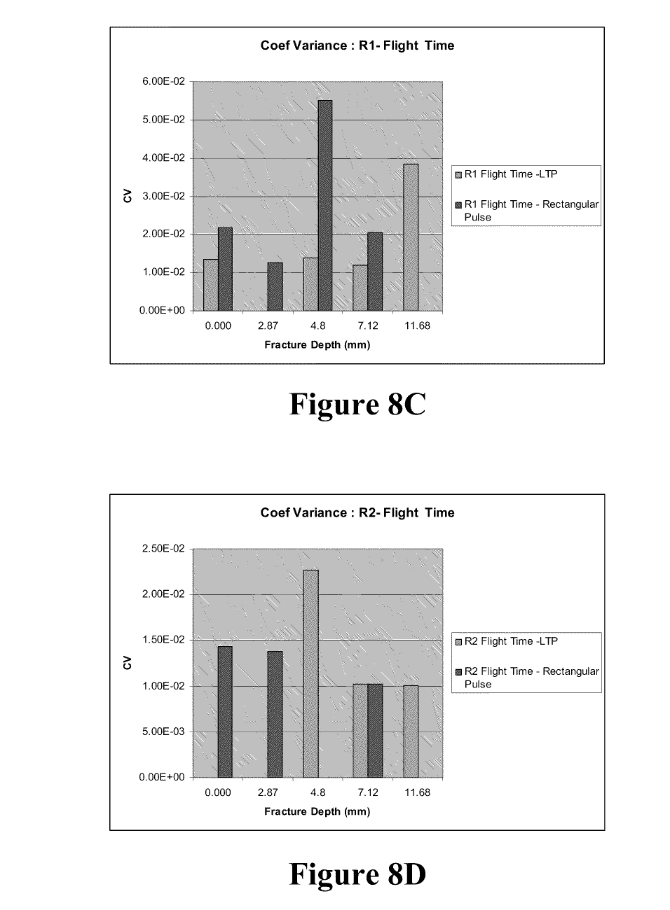

To evaluate the effectiveness of the drive pulses, an experiment was carried out on the sheep tibia using a transmitter and a receiver separated by 35 mm. The coefficients of variation are calculated for the flight time and the maximum amplitude of the acoustic signal captured at different fracture depth. As shown in FIGS. 8A-8D the frequency and the amplitude error involved in using RRP is much higher compared to LTP. FIGS. 8A-8D indicate that LTP produces more consistent results at different fracture depth.

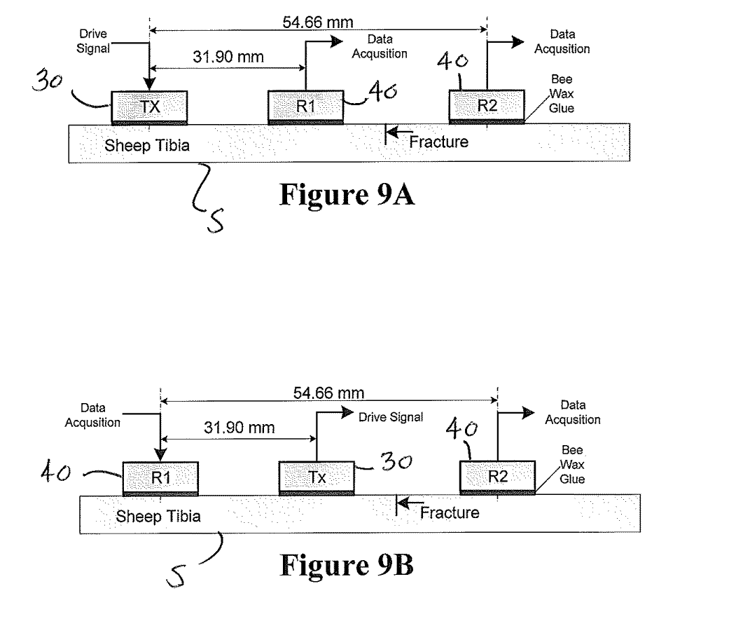

In a main experiment sheep tibia was used with five fracture conditions: 1) intact bone, 2) 2.87 mm fracture depth, 3) 4.80 mm fracture depth, 4) 7.12 mm fracture depth and 5) 11.68 mm fracture depth. At each depth, acoustic signals captured by R1 and R2 in both Setup 1 and Setup 2 were recorded and analyzed. Now referring to FIGS. 9A and 9B, the distance between transducers and their setup for this experiment is shown. Transducers Tx, R1 and R2 were mounted on the tibia using synthetic beeswax. LTP drive signals were used for all experiments. Acoustic signals captured by R1 and R2 in both Setup 1 and Setup 2 were recorded using a digital oscilloscope.

All of the recorded signals from R1 and R2 for both Setups were then analyzed and key parameters were calculated. The acoustic parameters of interest include:

Time of flight: the time taken by the acoustic energy to travel along the bone from transmitter to the receiver.

Maximum amplitude: value of the highest peak of the received acoustic signal.

Phase shift: the measurement of the phase changes in the received signal due to varying fracture depth.

Cross correlation: how the received signal with the presence of fracture correlates to the one without fracture.

### Flight Time Measurement

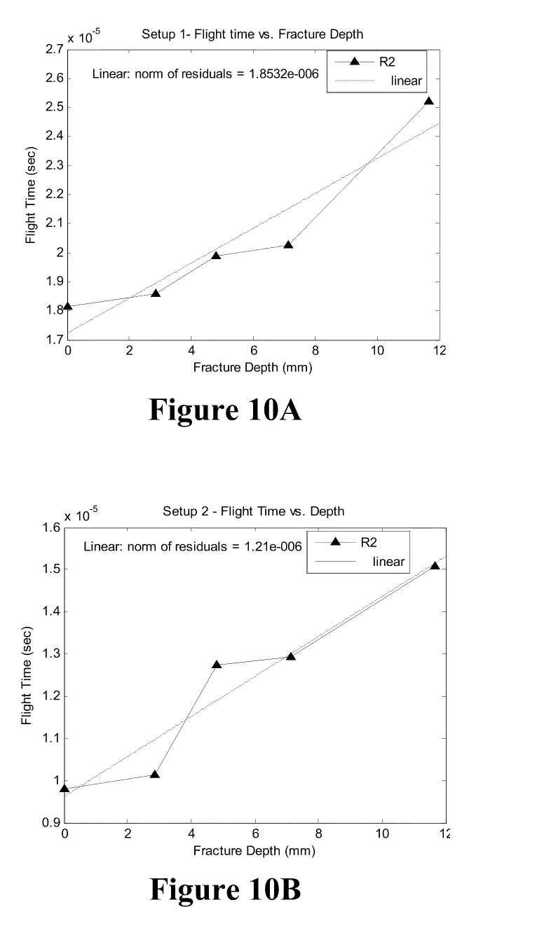

For each fracture depth, average values of the flight time at R1 and R2 in both Setups are shown in Table 1. FIGS. 10A and 10B depict plots of flight time of the received acoustic signal at R2 with respect to each fracture depth.

TABLE 1
Average flight time of the signal at R1 and R2 for both Setups

| | | | | |
|---|---|---|---|---|
| Depth (mm) | R1 (sec) | R1 (σ) (sec) | R2 (sec) | R2 (σ) (sec) |

Setup 1

| | | | | |
|---|---|---|---|---|
| 0 | 1.100e−5 | ±2.074e−21 | 1.813e−5 | ±2.309e−7 |
| 2.87 | 1.113e−5 | ±1.154e−07 | 1.860e−5 | ±0 |
| 4.80 | 1.107e−5 | ±1.154e−07 | 1.987e−5 | ±1.154e−7 |
| 7.12 | 1.100e−5 | ±2.074e−21 | 2.027e−5 | ±1.154e−7 |
| 11.68 | 1.127e−5 | ±1.154e−07 | 2.520e−5 | ±3.464e−7 |

Setup 2

| | | | | |
|---|---|---|---|---|
| 0 | 1.120e−5 | ±2.000e−7 | 9.800e−6 | ±0 |
| 2.87 | 1.120e−5 | ±0 | 1.013e−5 | ±1.154e−7 |
| 4.80 | 1.127e−5 | ±1.154e−7 | 1.273e−5 | ±1.154e−7 |
| 7.12 | 1.120e−5 | ±2.000e−7 | 1.293e−5 | ±1.154e−7 |
| 11.68 | 1.120e−5 | ±0 | 1.507e−5 | ±4.618e−7 |

FIGS. 10A and 10B show that the flight time of the acoustic signal for R2, which is the receiver mounted after the fracture for both Setups, undergoes an increase in its value with respect to the fracture depth. In both Setups, the flight time values for R1 do not change significantly with change in fracture depth, confirming no effect of fracture on that section of the bone. Setup 2 produces a more linear trend with a residue norm of 1.21 microseconds vs. 1.85 microseconds for Setup 1. The increase in flight time of acoustic signal with respect to the fracture depth indicates that the characteristic path of the acoustic waveform is being altered by the presence of a discontinuity in material density of the bone: deeper fractures results in longer equivalent pathways of acoustic signal and therefore longer flight time.

### Amplitude Measurement

Table 2 contains the averaged maximum amplitudes of recorded acoustic signal in both Setups.

TABLE 2
Average values of maximum amplitude of the signal at R1 and R2
for both setups.

| | | | | |
|---|---|---|---|---|
| Depth | | | | |
| (mm) | R1 | R1 (σ) | R2 | R2 (σ) |

Setup 1

| | | | | |
|---|---|---|---|---|
| 0 | 0.8282 | ±0.0020 | 0.5137 | ±0.0002 |
| 2.87 | 0.7705 | ±0.0029 | 0.4329 | ±0.0004 |
| 4.80 | 0.7464 | ±0.0005 | 0.3880 | ±0.0002 |
| 7.12 | 0.8236 | ±0.0020 | 0.3547 | ±0.0006 |
| 11.68 | 0.7532 | ±0.0005 | 0.2471 | ±0.0010 |

Setup 2

| | | | | |
|---|---|---|---|---|
| 0 | 0.8441 | ±0.0046 | 1.680 | ±0.0005 |
| 2.87 | 0.8040 | ±0.0049 | 1.520 | ±0.1149 |
| 4.80 | 0.8141 | ±0.0030 | 1.626 | ±0.0012 |
| 7.12 | 0.8177 | ±0.0025 | 1.303 | ±0.0092 |
| 11.68 | 0.7692 | ±0.0005 | 0.675 | ±0.0025 |

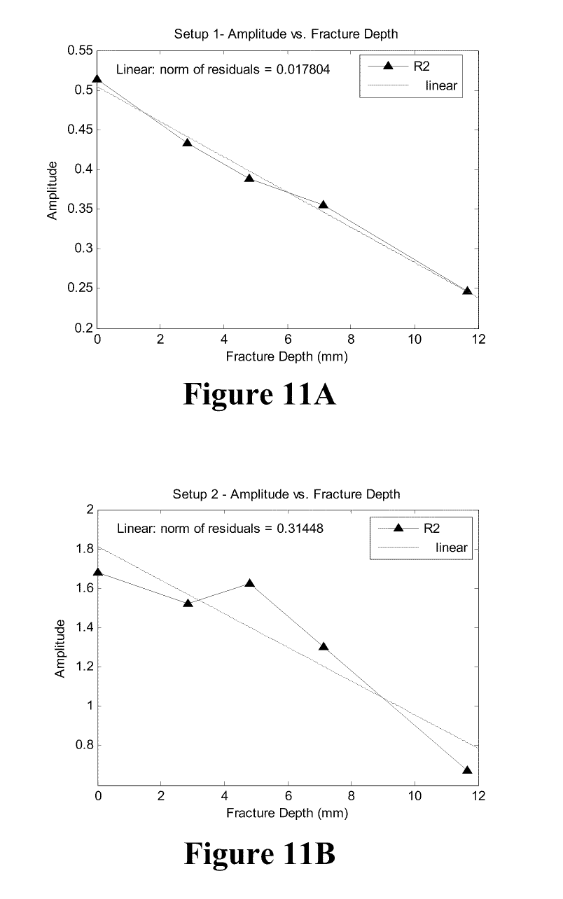

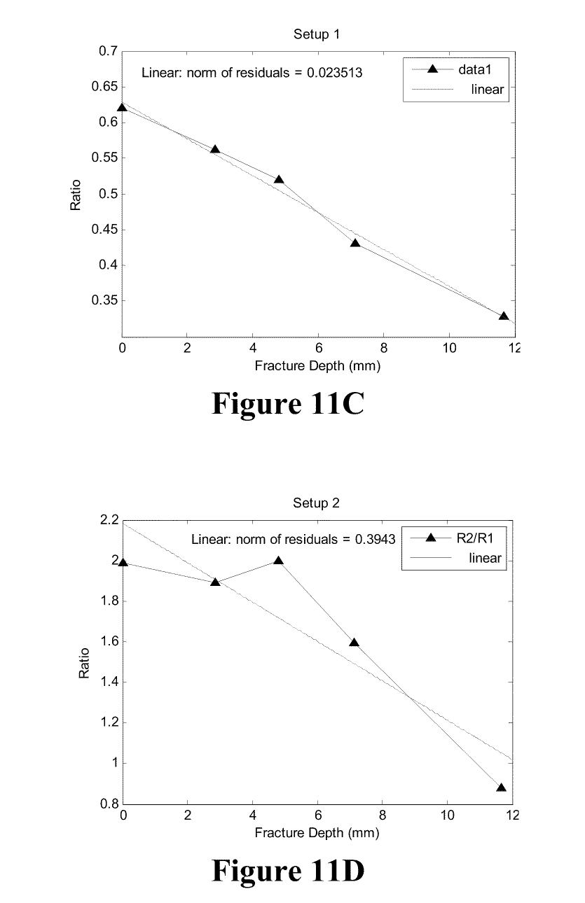

FIGS. 11A-11D show the changes occurring to the peak amplitude values of the recorded signal with respect to the fracture depth. The maximum amplitude for the acoustic signal captured by R2 in both Setups undergoes a linear drop as the fracture depth increases. Linear curve fitting for amplitude vs. fracture depth yields a lower residual value for Setup 1 (0.0178) than for Setup 2 (0.3943). In both cases, such rather linear characteristic confirms the high sensitivity of maximum amplitude with respect to fracture depth. The drop in maximum amplitude for receivers placed after the fracture indicates the increase in attenuation coefficient of the bone. Increase in fracture depth increases the area of discontinuity in bone which obstructs the clean transfer of acoustic energy through the medium and so maximum amplitude of the acoustic signal experiences a drop. Maximum amplitude of acoustic signal captured at R1 shows minor fluctuations well within experimental tolerances.

### Phase Measurement

Phase measurement provides an alternative characterization of changes in the signal shape due to fracture. Phase shifts between each output captured by R2 for both Setups for the five cut depths were calculated. The signal recorded on intact bone was used as the reference to calculate the phase shift. Tables 3 and 4 contain the values of phase shift in degrees. Table 3 shows a steady increase in phase angle for output captured in Setup 1 but output captured in Setup 2 fails to follow the same trend. The short distance between transmitter and R2 in Setup 2 may be responsible for this disturbance in linear trend of phase angle seen in Setup 1. Since the distance is short between the two transducers the reflection waves are more likely to be detected along with leading edge. This multi-pass situation might be responsible for creating interference in data and creating the disturbance in trend. From Tables 3 and 4 it can also be inferred that phase angle measurement loses its sensitivity as the fracture depth increases.

TABLE 3
Phase shift values for acoustic signal captured by R2 in setup 1

| Depth (mm) | Highest Peak Time | Phase Angle |
|---|---|---|
| 0 | 5.56E−05 | 0 |
| 2.87 | 5.60E−05 | 23.2 |
| 4.80 | 5.62E−05 | 34.8 |
| 7.12 | 5.64E−05 | 46.5 |
| 11.68 | 5.64E−05 | 46.5 |

TABLE 4
Phase shift values for acoustic signal captured by R2 in setup 2

| Depth (mm) | Highest Peak Time | Phase Angle |
|---|---|---|
| 0 | 4.76E−05 | 0 |
| 2.87 | 4.80E−05 | 21.2 |
| 4.80 | 4.78E−05 | 10.6 |
| 7.12 | 4.82E−05 | 31.8 |
| 11.68 | 4.82E−05 | 31.8 |

### Correlation Measurement

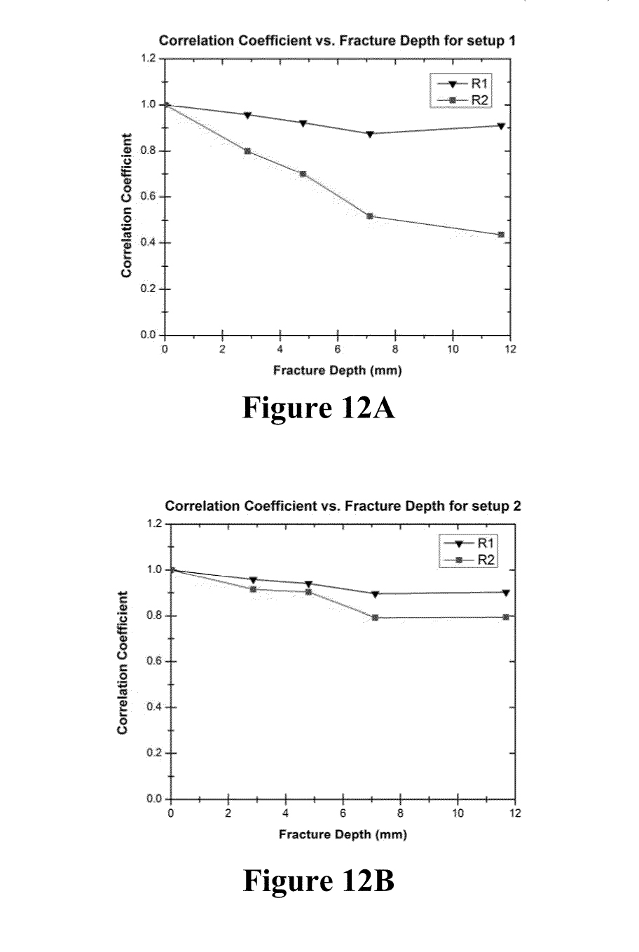

Correlation analysis was carried out on the acoustic signals acquired from R1 and R2 for the five cut depths. With the acoustic signals captured from R1 and R2 on the intact bone as the reference signals, correlation analyses were applied on the received signals at various cut depths in Setup 1 and Setup 2. The resultant correlation coefficients, indicating how the received signals from various cut depths correlate to the reference value, are listed in Tables 5 and 6 and plotted in FIGS. 12A and 12B.

TABLE 5
Correlation coefficients of acoustic signals captured in Setup1

| | |
|---|---|
| | Correlation |
| Fracture | Coefficient |

| Depth (mm) | R1 | R2 |
|---|---|---|
| 0 | 1 | 1 |
| 2.87 | 0.9565 | 0.7987 |
| 4.80 | 0.9217 | 0.6996 |
| 7.12 | 0.8746 | 0.5169 |
| 11.68 | 0.9094 | 0.4364 |

TABLE 6
Correlation coefficients of acoustic signals captured in Setup 2

| | |
|---|---|
| | Correlation |
| Fracture | Coefficient |

| Depth (mm) | R1 | R2 |
|---|---|---|
| 0 | 1 | 1 |
| 2.87 | 0.9576 | 0.9145 |
| 4.80 | 0.9397 | 0.9023 |
| 7.12 | 0.8958 | 0.7923 |
| 11.68 | 0.9020 | 0.7944 |

Now referring to FIGS. 10A and 10B, it can be seen observed that in both Setups for shallow fracture, the flight time exhibits relatively low sensitivity so that proper detection of small fractures just by using flight time of acoustic signal is difficult. On the other hand, maximum amplitude vs. fracture plots (FIGS. 11A and 11B) indicate steady and high sensitivity for all fracture depths. However, maximum amplitude is sensitive to mount condition and the presence of soft tissues. Therefore, a careful selection of each signal parameter by an algorithm allows quantifying the fracture characteristics accurately. When the fracture depth is small, the flight time of the acoustic output can be used in conjunction with the maximum amplitude and the phase shift of the acoustic output which supports the low sensitivity of flight time in shallow cuts. Similarly, if the fracture is deep, the flight time of acoustic output along with its maximum amplitude would be the ideal parameters. This would eliminate the low sensitivity of phase angle in deeper fractures. Correlation analysis shows consistent and sensitive detection and may be used in conjunction with the other three acoustic parameters.

As can be seen, behavioral patterns of different signal parameters such as flight time, maximum amplitude, phase angle, and correlation are useful indicators of the depth of a fracture and in accordance with the methods disclosed herein may be used in quantitative ultrasound bone fracture detection. It is shown herein that the described techniques, including those using LTP technology, make possible using ultrasound to detect small defects in bone which are missed by normal conventional fracture detection methods. An ultrasound device as described herein to detect bone defects quantifies the results, eliminating the need of a skilled technician to interpret the results.

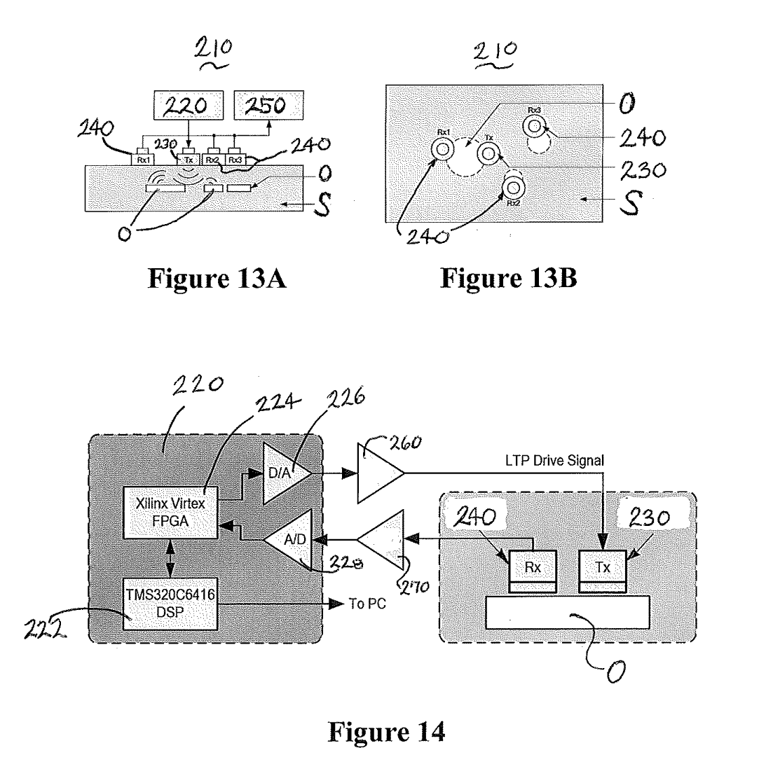

Now referring to FIGS. 13A and 13B, an alternate embodiment of the present invention includes a tumor detector system 210 having a signal generator 220, transmitter 230, receivers 240, and analyzer 250. The same exemplary components described hereinabove for the bone fracture detector may be employed in this embodiment. The transmitter 230 and receivers 240 are positioned on the biological tissue S to be analyzed. O represents an object such as a tumor. The transmitter 230 delivers sonic energy to the biological tissue S. The transmitter 230 may emit bursts of ultrasonic signals, continuous signals, or random noise. The presence of an object O, such as a tumor, having different density/features than the surrounding biological tissue S, alters the transmission characteristics of the sonic energy. In this way the transmitter 230 “illuminates” the tumor with ultrasonic energy while receivers 240 pick up the reflected signals. It is also possible to align the receiver 240 and transmitter 230 so that the receiver 230 picks up the transmitted signal as well.

In accordance with various embodiments the receiver(s) 240 and transmitter(s) 230 may be fixed or able to slide on the surface of the soft tissue so that different scan directions can be made with the combined data to construct a three-dimensional view of the tumor's location/size. In a preferred embodiment the system 210 is operated similar to a stethoscope. The transmitter 230 is deployed in a few fixed locations while one or more receivers 240 are moved to pick up acoustic signals at different locations.

Now referring to FIG. 14 a block diagram of an embodiment of the system 210 is shown including a transmitter 230, a receiver 240, and signal generator 220 having a digital signal processor (DSP) 222 with field programmable gate array (FPGA) 224, and amplifiers 260 and 270. Preferably a high speed digital signal processing system is connected to a PC to design and program the desired LTP drive signal and develop the algorithm for handling the received signal. Once this is done, the system 210 can be disconnected from the PC and run in stand-alone mode. While a drive signal such as a LTP drive signal is periodically output via a D/A converter 226 to drive the transmitter 230, the received signal is read into the DSP through the A/D converter 228 for further signal.

As will be apparent to those skilled in the art, the components used in the system 110 may be included in a hand-held device suitable for use as a breast tumor detector. With its low cost and compact size, this device can be made available to a large population segment, domestically and globally, that does not have adequate early detection capability.

### Experiments—Soft Tissue

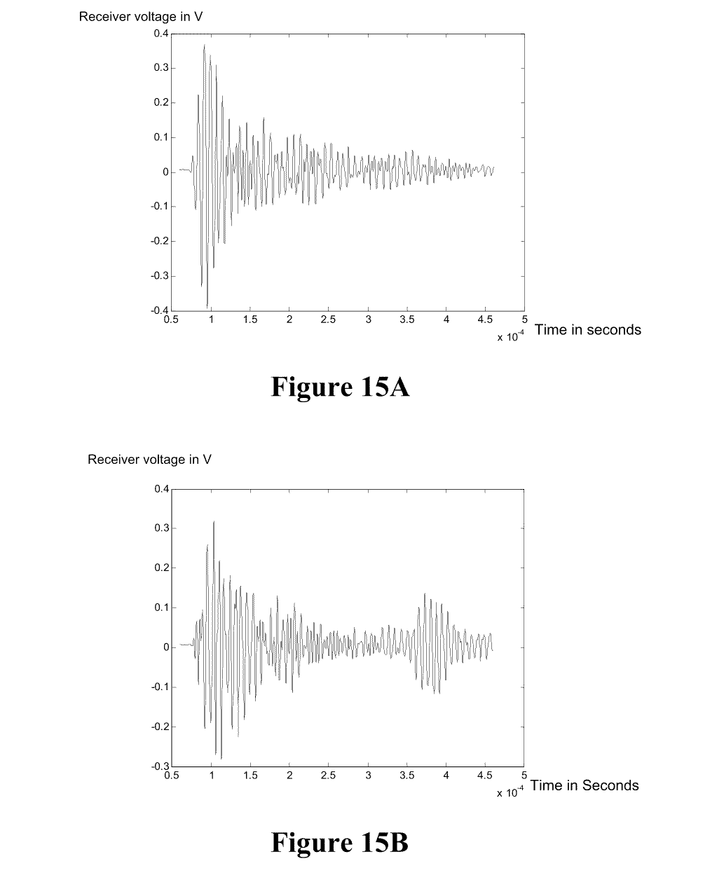

Now referring to FIGS. 15A and 15B, test data was generated using tumor nodules supplied by BluePhantom. The ultrasonic signal from phantom without tumor is shown in FIG. 15A while those with tumor are shown in FIG. 15B. The secondary echo for the RHS plot indicates the presence of a tumor in which the difference in mechanical impedance results in a secondary echo. By placing the receiver on different position and calculating flight times, the absolute position of the tumor can be determined.

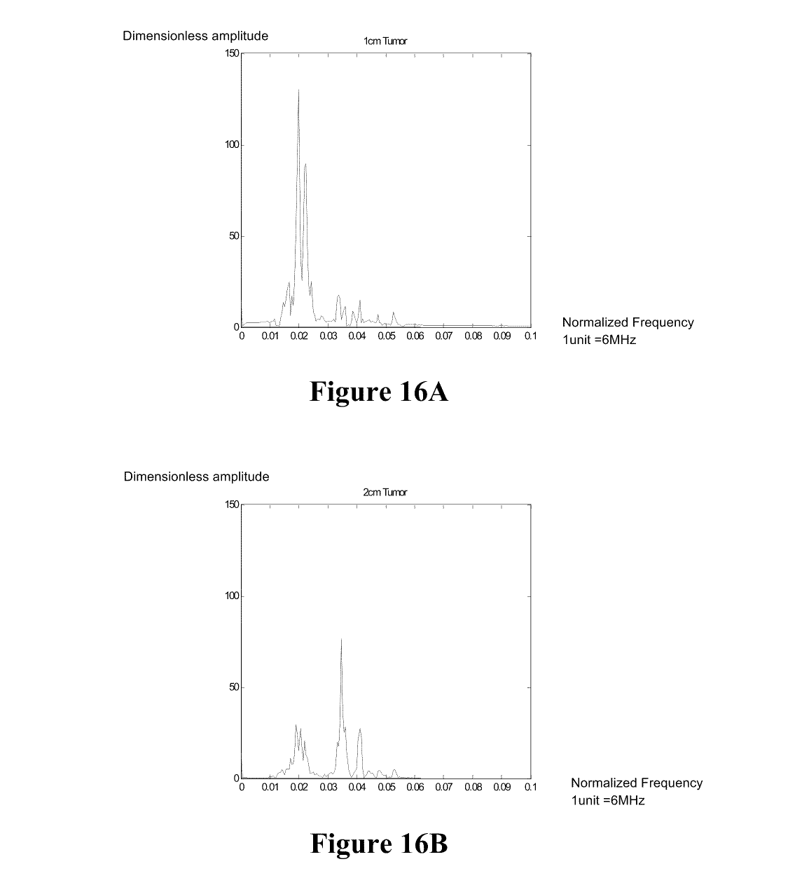

Now referring to FIGS. 16A and 16B, two phantoms with different feature sizes (10 cm—FIG. 16A; 2 cm—FIG. 16B) were tested and the spectra plotted. The change in frequency components provides secondary confirmation of the tumor feature sizes.

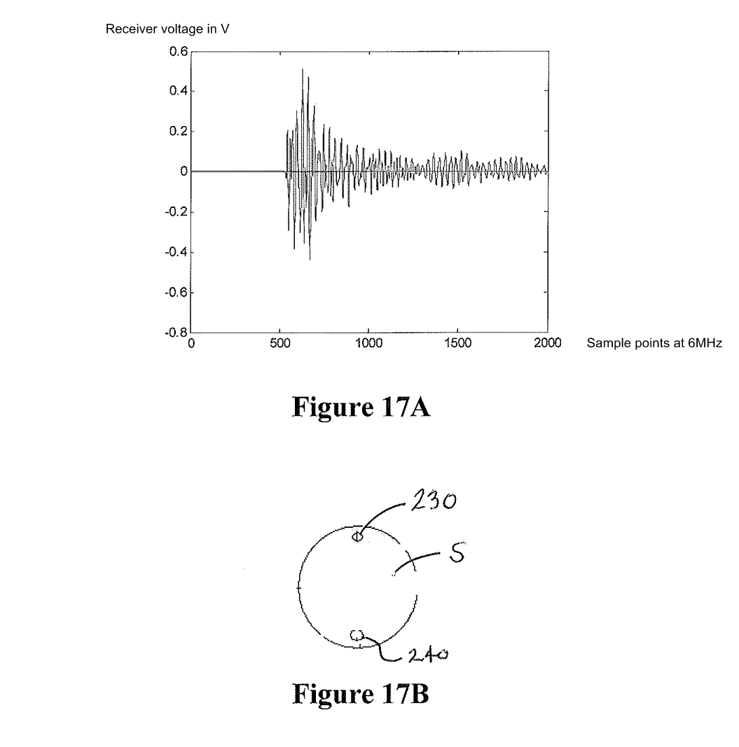

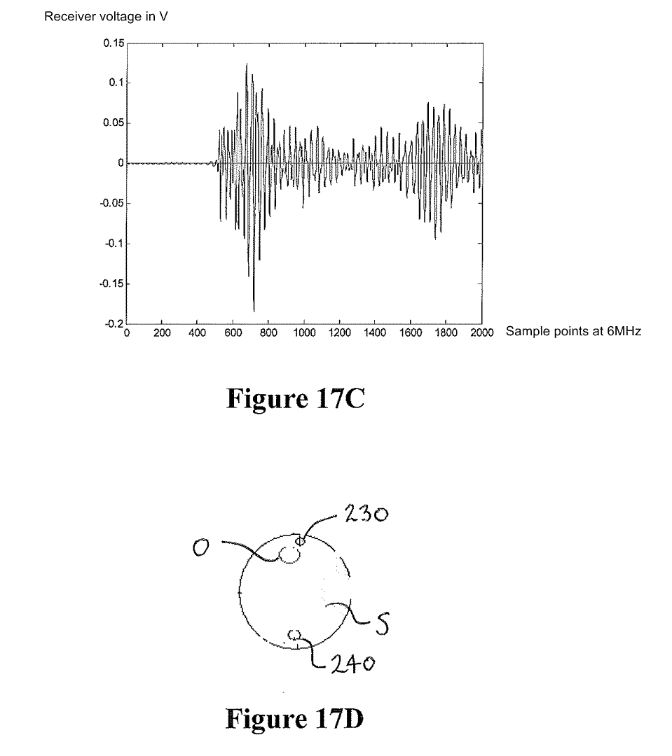

Now referring to FIGS. 17A-17D, an axial scan test was performed in accordance with one embodiment of the invention. Axial transmission of the ultrasonic signals was measured. FIG. 17A shows the signal without tumor. FIG. 17B is a diagrammatic depiction of tissue S without tumor and transmitter 230 and receiver 240 location. FIG. 17C shows the signal with tumor O. When a tumor O is present, near the transmitter as shown in FIG. 17D, a second echo is evident in the acoustic signal.

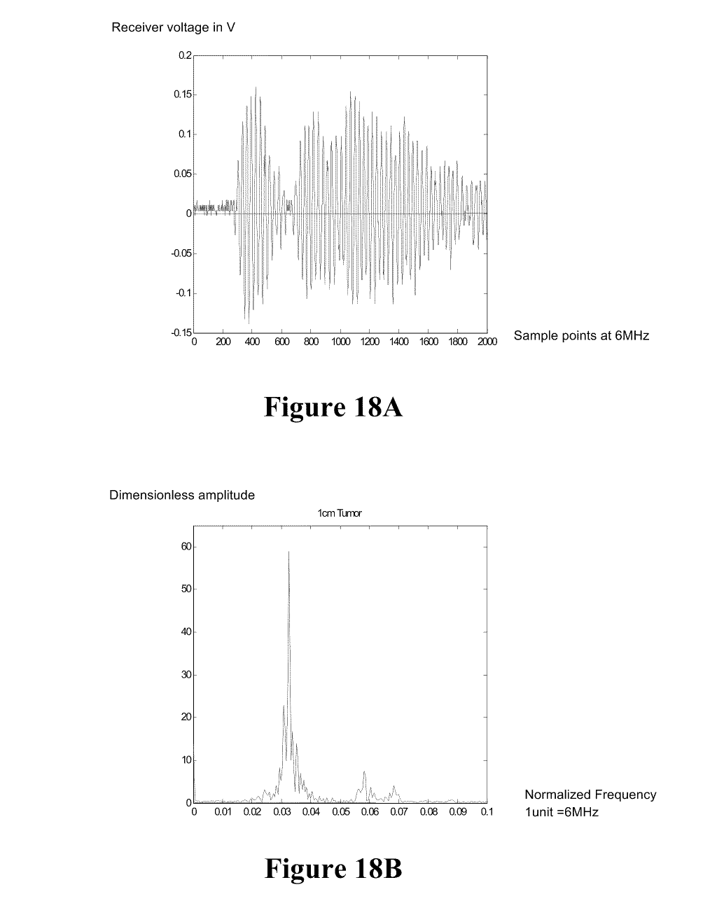

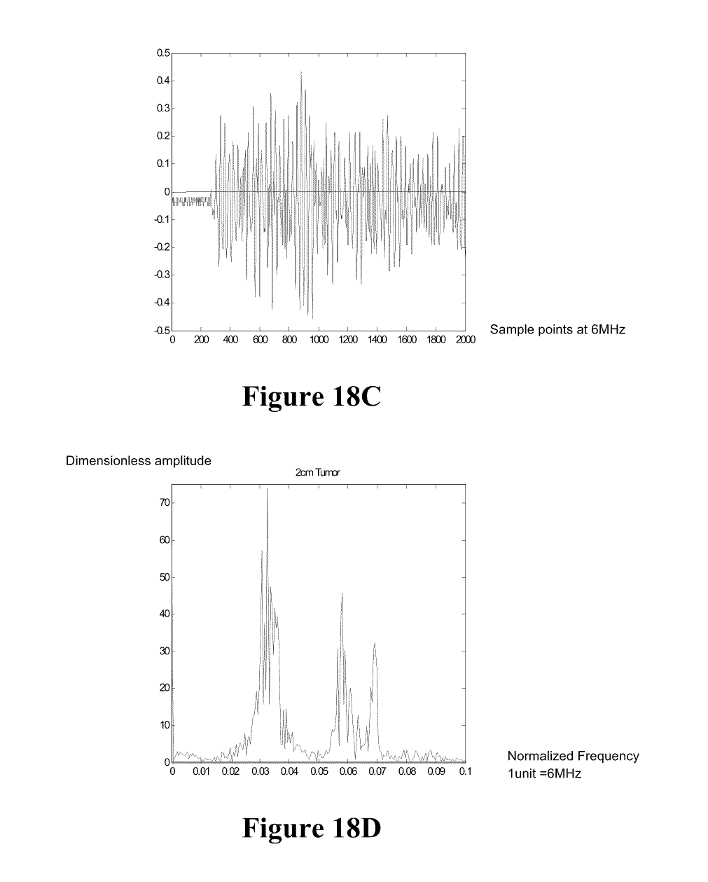

Now referring to FIGS. 18A-18D, the present invention may be used to feature size resolution. The system can be used to differentiate feature size due to reflective characteristics. FIGS. 18A and 18B show signal characteristics of 1 cm features, and FIGS. 18C and 18D show signal characteristics of 2 cm features. From measurement of the spectra, different feature sizes can be readily differentiated.

Although the systems and methods of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

All references cited herein are incorporated by reference herein in their entireties.

What is claimed is:

1. A system for analyzing biological tissue using ultrasound comprising a signal generator capable of generating a low transient pulse signal, a transmitter, at least one receiver and a signal analyzer.

2. A system according to claim 1 operable to detect and/or monitor structural damage and/or defects in bone.

3. A system according to claim 1 operable to detect and/or monitor a tumor in soft tissue.

4. A system according to claim 1 comprising a Quantitative Ultrasound System (QUS).

5. A system according to claim 1 comprising more than one receiver.

6. A system according to claim 1 wherein the signal analyzer is an oscilloscope.

7. A system according to claim 1 further comprising a data store operable to capture time and frequency domain parameters of a received signal.

8. A system according to claim 1 further comprising a computer programmed to analyze data selected from flight time, maximum amplitude, phase change, and correlation analysis of the received acoustic signal.

9. A method of analyzing biological tissue comprising generating a low transient pulse signal to a transmitter, applying the transmitter and at least one receiver to a body structure containing the region of biological tissue to be analyzed, transmitting the signal to the biological tissue to be analyzed, and transmitting a received signal to a signal analyzer.

10. A method according to claim 9 wherein the biological tissue to be analyzed is bone.

11. A method according to claim 9 wherein the biological tissue to be analyzed is soft tissue.

12. A method according to claim 9 further comprising interpreting time and frequency domain of the signal.

13. A method according to claim 10 comprising positioning the transmitter and the at least one receiver axially along long bones covered by a thin layer of soft tissue.

14. A method according to claim 10 comprising applying the transmitter and at least one receiver in positions to allow the capture of ultrasound surface waves propagating along the bone axis, to permit the analysis of the bone cortex.

15. The method according to claim 9 further comprising evaluating one or more signal parameters selected from amplitude, RMS energy, and flight time.

16. The method according to claim 10 comprising analyzing at least two parameters selected from flight time, maximum amplitude, phase change, and correlation analysis of the received acoustic signal.

17. The method according to claim 10 comprising placing the transmitter and at least one receiver to provide at least one reference reading which is unaffected by a fracture and at least one reading which is affected by the fracture, analyzing behavioral patterns of the signal parameters flight time, maximum amplitude, phase change, and correlation analysis of the received acoustic signal, and generating an algorithm which determines the fracture condition by comparing signal parameters from the first reading to signal parameters of the second reading.

18. The method according to claim 11 comprising analyzing a biological tissue for the presence of a tumor.

19. The method according to claim 11 comprising analyzing ultrasonic signals numerically to identify abnormal features of soft tissue.

20. The method according to claim 19 comprising analyzing signals transmitted through or reflected from an abnormal tissue for scattering, flight time and changes in spectral characteristics selected from frequency dependent magnitude and phase.

21. A system for analyzing animal soft tissue using ultrasound comprising a signal generator, a transmitter, at least one receiver and a signal analyzer.

22. A system according to claim 21 wherein the signal generator is capable of generating a low transient pulse signal 3.

23. A system according to claim 21 operable to detect and/or monitor a tumor in soft tissue.

24. A system according to claim 21 comprising a Quantitative Ultrasound System (QUS).

25. A system according to claim 1 comprising more than one receiver.

26. A system according to claim 1 wherein the signal analyzer is an oscilloscope.

27. A system according to claim 1 further comprising a data store operable to capture time and frequency domain parameters of a received signal.

28. A system according to claim 1 further comprising a computer programmed to analyze data selected from flight time, maximum amplitude, phase change, and correlation analysis of the received acoustic signal.

29. A method of analyzing animal soft tissue comprising generating signal to a transmitter, applying the transmitter and at least one receiver to a body structure containing the region of biological tissue to be analyzed, transmitting the signal to the soft tissue to be analyzed, and transmitting a received signal to a signal analyzer.

30. A method according to claim 29 wherein the signal generated is a low transient pulse signal.

31. A method according to claim 29 further comprising interpreting time and frequency domain of the signal.

32. The method according to claim 29 further comprising evaluating one or more signal parameters selected from amplitude, RMS energy, and flight time.

33. The method according to claim 29 comprising analyzing at least two parameters selected from flight time, maximum amplitude, phase change, and correlation analysis of the received acoustic signal.

34. The method according to claim 29 comprising analyzing a biological tissue for the presence of a tumor.

35. The method according to claim 29 comprising analyzing ultrasonic signals numerically to identify abnormal features of soft tissue.

36. The method according to claim 29 comprising analyzing signals transmitted through or reflected from an abnormal tissue for scattering, flight time and changes in spectral characteristics selected from frequency dependent magnitude and phase.
